(19) United States
(12) Patent Application Publication
(10) Pub. No.: US 2020/0375532 A1
(43) Pub. Date: Dec. 3, 2020

(54) HYDROTHERMAL SENSING DEVICE

(71) Applicant: SENSEHydro LLC, Chandler, AZ (US)

(72) Inventors: Rachna Nath, Chandler, AZ (US); Sohani Sandhu, Gilbert, AZ (US); Kaitlyn Lai, Chandler, AZ (US); Omina Nematova, Chandler, AZ (US); Abraham Troop, Mesa, AZ (US); Ivan-Alexander Kroumov, Chandler, AZ (US); Nealin Banerjee, Chandler, AZ (US); Diya Nath, Chandler, AZ (US); Jacob Kaufman Warner, Chandler, AZ (US); Daniel Wu, Chandler, AZ (US)

(21) Appl. No.: 16/882,933

(22) Filed: May 26, 2020

Related U.S. Application Data
(60) Provisional application No. 62/852,703, filed on May 24, 2019.

(51) Int. Cl. A61B 5/00; A61B 5/0205; A41D 20/00; A42B 1/22; A41D 27/28; A41D 31/12; A41D 1/00

(52) U.S. Cl. CPC A61B 5/4875; A61B 5/02055; A61B 5/6803; A41D 20/00; A61B 5/6831; A61B 5/6815; A61B 5/02438; A41D 27/28; A41D 31/125; A61B 5/6824; A61B 5/002; A41D 1/002; A42B 1/22

(57) ABSTRACT

An hydrothermal sensing device wherein the hydrothermal sensing device comprises at least one layer wherein the at least one layer is a reflective layer, a mesh layer, a semi-rigid layer and a wicking layer wherein the reflective layer can be attached to the mesh layer, and the mesh layer can be attached to the semi-rigid layer and the wicking layer can be removably attached to the semi-rigid layer. The semi-rigid layer can further comprise a band wherein the band can comprise a printed circuit board having at least one sensing module, a microcontroller, a communications module, and power source. The reflective layer is porous and UV resistant.

### CROSS-REFERENCE TO RELATED APPLICATIONS

This application claims benefit from currently pending U.S. Provisional Application No. 62/852,703 titled “Enhanced Personal Protection Device in the Form of a Hydrothermal-Sensor Head Covering” and having a filing date of May 24, 2019, all of which is incorporated by reference herein.

### BACKGROUND OF THE INVENTION

### Field of the Invention

The present invention relates to a head covering for detecting a user's vitals, and more particular a head covering device that actively monitors a user's pulse, temperature and elevation and then relays the user's information to a portable computing device.

### Description of Related Art

Various types of health monitoring systems exist in the art. For example, there are some clothes or shoes that promote air circulation but generally do not cool the user down in extreme high heat index conditions. Working in extreme conditions can leave the user susceptible to dehydration and heat stroke. Wicking materials have been developed to help draw moisture away from the body keeping the user cool for longer periods of time. In addition, wicking materials are breathable thus allowing for air to flow to the user's body. However, this type of material alone does not alert or notify the user if they have lost too much fluids and are approaching heat stroke and dehydration. Products have been developed to further cool the user for longer periods of times such as the Arctic Hat. The Arctic Hat allows the user to moisten the inside of the hat with water and relies on evaporative cooling to keep the user's body temperature up to 20 degrees Fahrenheit cooler than the ambient temperature. Additional products are similar to the Arctic Hat but are made to only wrap or tie around the user's head. However, these types of products do not incorporate any technology to alert the user or a third party the user's status.

Other types of monitoring devices exist on the market today which are usually attached to a user's wrist or other parts of the human body. However, these devices cannot properly determine whether the individual is approaching dehydration or heat stroke, nor can they provide a cooling effect to the user allowing for the user to stay cooler for longer periods of time during extreme temperatures while exercising, working, hiking, or doing other activities outdoors. These existing monitoring devices are limited in function wherein they do not alert the user or the user's guardian or a third party that the user is getting closer to heat stroke and dehydration.

Therefore, there is a need for a device that keeps the user's cool in extreme temperatures and can alert the user or a third party if the user is approaching or has heat stroke and/or dehydration.

### SUMMARY OF THE INVENTION

Aspects disclosed herein relates to a Hydrothermal Sensing Device. A hydrothermal sensing device, wherein the hydrothermal sensing device comprises at least one layer wherein the at least one layer is a reflective layer, a mesh layer, a semi-rigid layer and a wicking layer wherein the reflective layer can be attached to the mesh layer, and the mesh layer can be attached to the semi-rigid layer and the wicking layer can be removably attached to the semi-rigid layer. The semi-rigid layer can further comprise a band wherein the band can comprise a printed circuit board having at least one sensing module, a microcontroller, a communications module, and power source. The reflective layer is porous and UV resistant. The mesh layer further comprises a brim and an adjustable snap closure. The at least one sensing module can be a thermal sensor, an accelerometer, and a pulse meter. The communications module is Bluetooth Low Energy or Wi-Fi. The wicking layer can retain fluid and wick away fluid from the user's body.

A method for detecting heat stroke and dehydration using a hydrothermal sensing device and a portable computing device comprising inputting a user's information such as age, weight, height, gender, activity level, baseline temperature, baseline pulse rate, and emergency contact information into the portable computing device. Monitoring and collecting a user's temperature, a pulse rate, and acceleration using the hydrothermal sensing device. Transmitting the temperature, pulse rate and acceleration from the hydrothermal sensing device to the portable computing device. Analyzing the temperature, pulse rate and acceleration from the hydrothermal sensing device and comparing that data to the user's information. Alerting user or a third party if the temperature, pulse rate or acceleration goes above a set threshold. Continuing monitoring if thresholds are not met. The temperature threshold is at least 102 degrees Fahrenheit. The pulse rate threshold is at least 20 beats per minute greater than the user's baseline pulse rate. The acceleration is the change in height or elevation of the user.

Additional features and advantages of the present specification will become apparent to those skilled in the art upon consideration of the following detailed description of the illustrative embodiment exemplifying the best mode of carrying out the invention as presently perceived.

Aspects and applications of the invention presented here are described below in the drawings and detailed description of the invention. Unless specifically noted, it is intended that the words and phrases in the specification and the claims be given their plain, ordinary, and accustomed meaning to those of ordinary skill in the applicable arts. The inventors are fully aware that they can be their own lexicographers if desired. The inventors expressly elect, as their own lexicographers, to use only the plain and ordinary meaning of terms in the specification and claims unless they clearly state otherwise and then further, expressly set forth the. Absent such clear statements of intent to apply a “special” definition, it is the inventor's intent and desire that the simple, plain, and ordinary meaning to the terms be applied to the interpretation of the specification and claims.

The inventors are also aware of the normal precepts of English grammar. Thus, if a noun, term, or phrase is intended to be further characterized, specified, or narrowed in some way, then such noun, term, or phrase will expressly include additional adjectives, descriptive terms, or other modifiers in accordance with the normal precepts of English grammar. Absent the use of such adjectives, descriptive terms, or modifiers, it is the intent that such nouns, terms, or phrases be given their plain, and ordinary English meaning to those skilled in the applicable arts as set forth above.

Further, the inventors are fully informed of the standards and application of the special provisions of 35 U.S.C. § 112 (f). Thus, the use of the words “function,” “means” or “step” in the Detailed Description or Description of the Drawings or claims is not intended to somehow indicate a desire to invoke the special provisions of 35 U.S.C. § 112 (f), to define the invention. To the contrary, if the provisions of 35 U.S.C. § 112 (f) are sought to be invoked to define the inventions, the claims will specifically and expressly state the exact phrases “means for” or “step for”, and will also recite the word “function” (i.e., will state “means for” performing the function of molding a fishing lure, without also reciting in such phrases any structure, material or act in support of the function. Thus, even when the claims recite a “means for performing the function of molding a fishing lure, step for performing the function of molding a fishing lure,” if the claims also recite any structure, material or acts in support of that means or step, or that perform the recited function, then it is the clear intention of the inventors not to invoke the provisions of 35 U.S.C. § 112 (f). Moreover, even if the provisions of 35 U.S.C. § 112 (f) are invoked to define the claimed inventions, it is intended that the inventions not be limited only to the specific structure, material or acts that are described in the preferred embodiments, but in addition, include any and all structures, materials or acts that perform the claimed function as described in alternative embodiments or forms of the invention, or that are well known present or later-developed, equivalent structures, material or acts for performing the claimed function.

Additional features and advantages of the present specification will become apparent to those skilled in the art upon consideration of the following detailed description of the illustrative embodiment exemplifying the best mode of carrying out the invention as presently perceived.

### BRIEF DESCRIPTION OF THE DRAWINGS

These and other features, aspects, and advantages of the present specification will become better understood with regard to the following description, appended claims, and accompanying drawings where:

FIG. 1 illustrates an isometric view of hydrothermal sensing device in accordance to one, or more embodiments;

FIG. 2 illustrates a front view of hydrothermal sensing device in accordance to one, or more embodiments;

FIG. 3 illustrates a back view of hydrothermal sensing device in accordance to one, or more embodiments;

FIG. 4 illustrates a side view of hydrothermal sensing device in accordance to one, or more embodiments;

FIG. 5 illustrates an exploded view of hydrothermal sensing device in accordance to one, or more embodiments;

FIG. 6 illustrates an isometric view of another embodiment of a hydrothermal sensing device in accordance to one, or more embodiments;

FIG. 7 illustrates an isometric view of another embodiment of a hydrothermal sensing device in accordance to one, or more embodiments;

FIG. 8 illustrates a top view of another embodiment of a hydrothermal sensing device in accordance to one, or more embodiments; and

FIG. 9 illustrates a flow diagram of a hydrothermal sensing device in accordance to one, or more embodiments.

### DETAILED DESCRIPTION OF THE INVENTION

In the following description, and for the purposes of explanation, numerous specific details are set forth in order to provide a thorough understanding of the various aspects of the invention. It will be understood, however, by those skilled in the relevant arts, that the present invention may be practiced without these specific details. In other instances, known structures and devices are shown or discussed more generally in order to avoid obscuring the invention. In many cases, a description of the operation is sufficient to enable one to implement the various forms of the invention, particularly when the operation is to be implemented in software. It should be noted that there are many different and alternative configurations, devices and technologies to which the disclosed inventions may be applied. The full scope of the inventions is not limited to the examples that are described below.

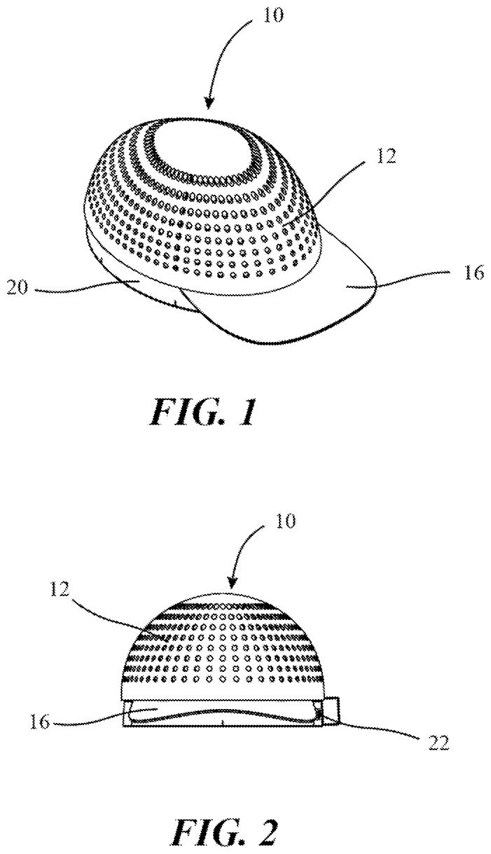

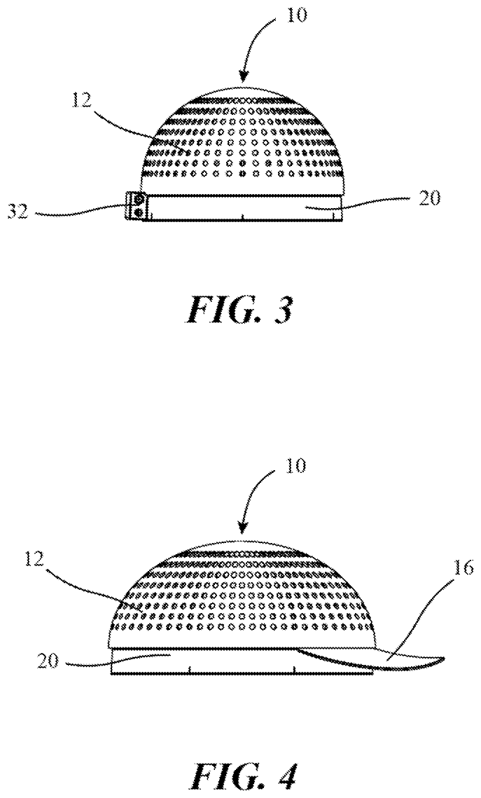

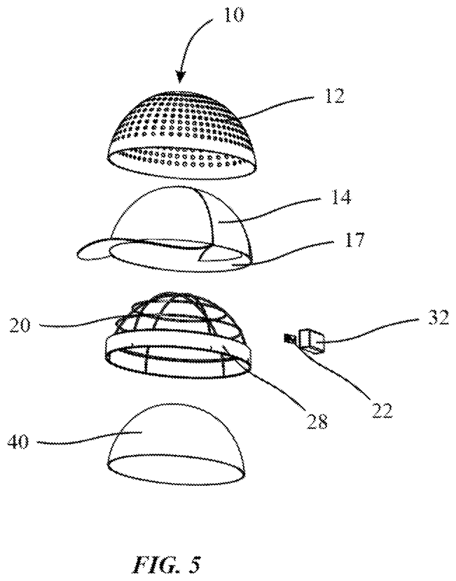

Referring to FIG. 1-5, a hydrothermal sensing device is shown generally at 10. In a preferred embodiment, a hydrothermal sensing device 10 can comprise at least one layer wherein there can be a reflective layer 12, a mesh layer 14, a semi-rigid layer 20 and a wicking layer 40. The reflective layer 12 can comprise of a porous and reflective material such as polyester, nylon, fabric laminates, metallized fabric, dire metallized fabric, or the like. The reflective layer 12 can be the outer layer of the hydrothermal sensing device 10 and can reflect the sun's ultraviolet rays away from the user while still allowing for air flow. The reflective layer 12 can take on any shape that can be worn by the user such as the shape of the user's head, wrist, torso, arm, or the like. The reflective layer 12 can be attached to the mesh layer 14 wherein the mesh layer can further comprise a brim 16 and a snap closure 17 wherein the brim can protect the user's face from the sun and the snap closure can adjust to different user head sizes.

The mesh layer 14 can be manufactured from cotton, nylon, polyester, rayon, linen, silk, or the like. The brim 16 can have a rigid structure underneath the mesh material allowing for the brim to stay supported and protrude away from the user's head. The mesh layer 14 can allow for air flow to the user's head. In certain embodiments the brim 16 can be omitted. The non-limiting example of the embodied hydrothermal sensing device is shown take the shape of a hat, but the hydrothermal sensing device can be in the form of a wristband, headband, belt, earpiece or the like. Additionally, the hydrothermal sensing device 10 can include head coverings such as scarves (veils, hijabs, burqas, or the like), bonnets, turbans, helmets, hoods and hats in all forms. The hydrothermal sensing device 10 can cover a portion of the head.

The mesh layer 14 can be attached to the semi-rigid layer 20 by sewing, snaps, zippers adhesive, or the like. The semi-rigid layer 20 can further comprise a band 28 wherein the band can further comprise a printed circuit board (“PCB”) wherein the PCB can comprise a micro-controller, a communication module and one or more sensing modules. The PCB 22 can be a flexible PCB, or a solid PCB wherein the PCB can be connected to a power supply 32. The sensing module can be placed directly on the user's head, ear, or temple to monitor the user's information such as, but not limited to heart rate, temperature, respiratory rate, oxygen levels, electrodermal activity, blood pressure, ambient temperature, heat index, moisture levels, changes in body temperatures, changes in moisture levels, movement, unconsciousness, or the like. The sensing module can be more than one and any combination of the following sensors, a thermal sensor, accelerometer, infrared sensor, pulse meter, digital thermometer, or the like.

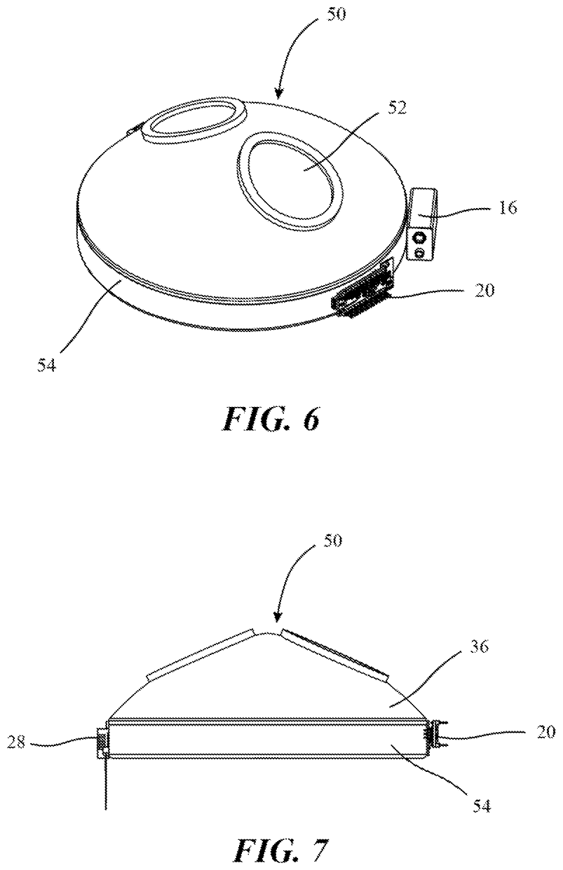

The communication module can relay the sensing module's information to a portable communication device such as a smart phone, tablet, or computer. The communication module can transmit via Bluetooth Low Energy (“BLE”), Wi-Fi, or any other suitable wireless communications module. In certain embodiments, an array of sensing modules can be placed around the inner perimeter of the band 28, or on the inner surface of the wireframe wherein the sensing modules can detect an array of temperatures throughout the user's head which can then be transmitted to the user or a third party's portable computing device where a thermal heat map of the user's head, or in other embodiments wrist, waist, ear, or the like can be displayed. The band 28 can further comprise a power supply 32 wherein the power supply can be coin-cell battery, nickel cadmium, nickel metal hydride, and lithium ion, or the like wherein the power supply can be recharged by solar panels 52 (as shown in FIG. 6), inductance charging, or wired charging. In certain embodiments the PCB can further comprise an audible buzzer and a light source such as light emitting diodes (“LED”), which can visually and audibly alert the user that heat stroke or dehydration is approaching when a portable computing device is not near the hydrothermal sensing device location.

The band 28 can be adjustable to fit a variety of sizes of the user's head, wrist, or other parts of the user's body and can be manufactured from any suitable material that keep its form or allow for electronics to be attached to it by hook and snaps, clips, snaps or the like. The band 28 can have a frame 38 and can have the same shape of the reflective layer 12 and the mesh layer 14. The frame 38 can be a nylon or plastic frame that can support the reflective layer 12 and the mesh layer 14 and help keep the hydrothermal sensing device's 10 shape. The frame 38 can be semi-rigid, flexible, rigid, or the like to allow the hydrothermal sensing device 10 keep its shape. The frame 38 can be attached to the band 28 by being sewn, glued, formed, or the like so that both the frame and the band can be one piece inside of the mesh layer 14 and the reflective layer 12.

The wicking layer 40 can be attached to the to the semi-rigid layer 20 by snaps, zipper or the like that can allow the wicking layer to be removed from the semi-rigid layer. The wicking layer 40 can be the same shape as semi-rigid layer 20, the mesh layer 14 and the reflective layer 12. The wicking layer 40 can be polyester, nylon, or and any fabric that has been treated with a solution to prevent water absorption and allows for the user's sweat to be wicked away from the user's body. The wicking layer 40 can provide an evaporative cooling effect on the user's head, such that when the wicking material is wet with water the temperature of the user's head decreases. In certain embodiments the wicking layer 40 can have at least one hole to allow for the sensing modules to access the user's head to allow for a more accurate reading directly from the user's skin. In certain embodiment the wicking layer 40 can be omitted.

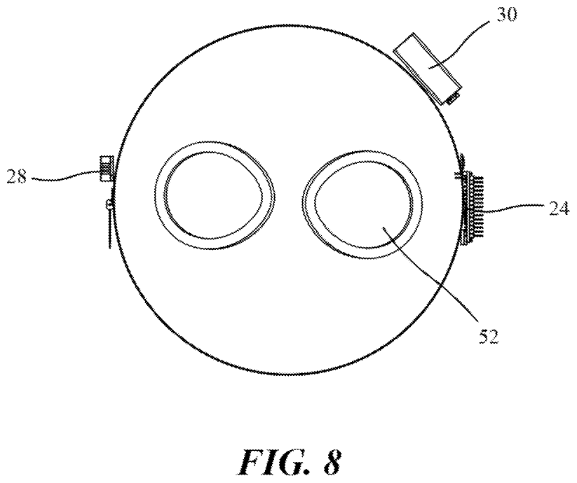

Referring to FIG. 6 through 8 another embodiment of a hydrothermal sensing device 50 wherein the hydrothermal sensing device can omit the reflective layer 12, mesh layer 14 and the semi-rigid layer keeping the wicking layer 40 having solar panels 52 allowing the power supply 30 to be recharged by the sun. In certain embodiments the wicking layer 40 can be omitted and just the band 28, PCB 22, sensing modules 28, and power supply 30 are included in the hydrothermal sensing device 50 allowing for the hydrothermal sensing device to be sized down or up to be placed around an individual's wrist, ear, head, torso, or the like.

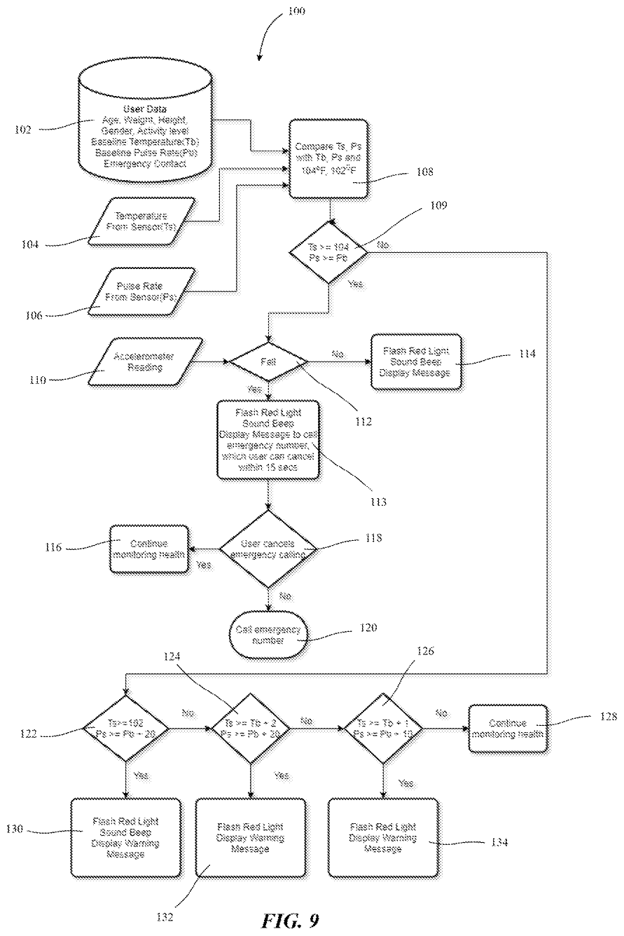

Referring to FIG. 9, a method for detecting heat stroke and dehydration using a hydrothermal sensing device 10 interacting with a portable computing device at 100. At 102 a user can input data such as age, weight, height, gender, activity level, baseline temperature, baseline pulse rate, and emergency contact information into the portable computing device. The activity level can be how many minutes of activity a day and how many days per week is the user active such as, 20-30 minutes, 3-4 days a week; 30-60 minutes, 5-6 days a week; and above 60 minutes, 7 days a week. At 104 temperature, 106 pulse rate and 108 accelerometer data is transmitted from the communications module from the hydrothermal sensing device 10 to the portable computing device wherein the portable computing device can compare the baseline temperature and baseline pulse rate with the data transmitted from the hydrothermal sensing device of the user's temperature and pulse rate. The temperature and pulse rate can be recorded and transmitted every at least every second, and the values can be averaged every 5 seconds. At 109 a comparison is made to determine whether the user's temperature is between 102-104 degrees Fahrenheit and whether the user's pulse rate is at least 20 beats per minute greater than baseline pulse rate. If the user's temperature reaches between 102-104 degrees Fahrenheit and the pulse rate increase by at least 20 beats the portable computing device will display a notice that the user is at risk of heat-related illness and you must drink water and go to a cooler place immediately.

At 112 the portable computing device analyzes the accelerometer reading, the temperature reading and the pulse rate of the user wherein the portable computing device determines whether the user height or elevation has changed, temperature range is above 104 degrees Fahrenheit, and pulse rate has increased from the user's baseline pulse rate. If the user height or elevation has drastically changed then at 114 the portable computing device can send a signal back to the portable computing device 10 and the portable computing device can flash the lights, audible alarm, and display a message on the portable computing device. At step 113 if the portable computing device detects a drastic change in height or elevation, pulse rate increase and the user's temperature above 104 degrees Fahrenheit then a timer will be set for at least 15 seconds where the user or a third party can cancel the alarm, if the user or third party does not cancel the alarm then at 120 an emergency number can be called, or at 118 the user cancel the emergency calling the hydrothermal sensing device and the portable computing device continue to monitor the user's health.

At 109 a comparison is made to determine whether the user's temperature is between 102-104 degrees Fahrenheit and whether the user's pulse rate is at least 20 beats per minute greater than baseline pulse rate, if the user's pulse rate and temperature is below 102 degrees Fahrenheit and below the pulse rate then the hydrothermal sensing device and the portable computing device continue to monitor the user's health and compares the user's temperature and if the user's temperature is at least 102 degrees Fahrenheit and the user's pulse rate is above twenty beats per minute from the baseline pulse rate, if the user exceeds the temperature and pulse rate then at 130 the portable computing device can send a signal back to the hydrothermal sensing device 10 and the hydrothermal sensing device can flash the lights, audible alarm, and the portable computing device can display a message to the user or a third party. If the user health does not exceed the temperature and pulse rate then at 124 if the user exceeds the baseline temperature by two degrees and pulse rate of at least 20 from the baseline pulse rate then at 132 the portable computing device can send a signal back to the hydrothermal sensing device 10 and the hydrothermal sensing device can flash the lights, audible alarm, and display a message on the portable computing device to the user or a third party. If the user health does not exceed the temperature and pulse rate then at 126 if the user exceeds the baseline temperature by one degrees and pulse rate of at least 10 from the baseline pulse rate then at 134 the portable computing device can send a signal back to the hydrothermal sensing device 10 and the hydrothermal sensing device can flash the lights, audible alarm, and display a message on the portable computing device to the user or third party. If the user still does not exceed the temperature and pulse rate threshold then the hydrothermal sensing device and portable computing device can continue to monitor the user's health.

A method for detecting heat stroke and dehydration using a hydrothermal sensing device and a portable computing device comprising inputting a user's information such as age, weight, height, gender, activity level, baseline temperature, baseline pulse rate, and emergency contact information into the portable computing device. Monitoring and collecting a user's temperature, a pulse rate, and acceleration using the hydrothermal sensing device. Transmitting the temperature, pulse rate and acceleration from the hydrothermal sensing device to the portable computing device. Analyzing the temperature, pulse rate and acceleration from the hydrothermal sensing device and comparing that data to the user's information. Alerting a user or a third party if the temperature, pulse rate or acceleration goes above a set threshold. Continuing monitoring if thresholds are not met. The temperature threshold is at least 102 degrees Fahrenheit. The pulse rate threshold is at least 20 beats per minute greater than the user's baseline pulse rate. The acceleration is the change in height or elevation of the user followed by inactivity.

It is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only and is not intended to limit the scope of the present disclosure, which is defined solely by the claims. Accordingly, embodiments of the present disclosure are not limited to those precisely as shown and described.

Certain embodiments are described herein, including the best mode known to the inventors for carrying out the methods and devices described herein. Of course, variations on these described embodiments will become apparent to those of ordinary skill in the art upon reading the foregoing description. Accordingly, this disclosure includes all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

What is claimed is:

1. A hydrothermal sensing device, wherein the hydrothermal sensing device comprises:
at least one layer wherein the at least one layer is a reflective layer, a mesh layer, a semi-rigid layer and a wicking layer wherein the reflective layer can be attached to the mesh layer, and the mesh layer can be attached to the semi-rigid layer and the wicking layer can be removably attached to the semi-rigid layer;
the semi-rigid layer can further comprise a band wherein the band can comprise a printed circuit board having at least one sensing module, a microcontroller, a communications module, and power source.

2. The device of claim 1, wherein the reflective layer is porous and UV resistant.

3. The device of claim 1, wherein the mesh layer further comprises a brim and an adjustable snap closure.

4. The device of claim 1, wherein the sensing modules are a thermal sensor, an accelerometer, and a pulse meter.

5. The device of claim 1, wherein the communications module is Bluetooth Low Energy or Wi-Fi.

6. The device of claim 1, wherein the wicking layer can retain fluid and wick away fluid from the user's body.

7. The device of claim 1, wherein printed circuit board is flexible.

8. The device of claim 1, wherein the hydrothermal sensing device is a hat, wristband, headband, belt, or earpiece.

9. A method for detecting heat stroke and dehydration using a hydrothermal sensing device and a portable computing device comprising:
inputting a user's information such as age, weight, height, gender, activity level, baseline temperature, baseline pulse rate, and emergency contact information into the portable computing device;
monitoring and collecting a user's temperature, a pulse rate, and acceleration using the hydrothermal sensing device;
transmitting the temperature, pulse rate and acceleration from the hydrothermal sensing device to the portable computing device;
analyzing the temperature, pulse rate and acceleration from the hydrothermal sensing device and comparing that data to the user's information;
alerting user or a third party if the temperature, pulse rate or acceleration goes above a set threshold; and
continuing to monitor if thresholds are not met.

10. The method of claim 9, wherein the temperature threshold is at least 102 degrees Fahrenheit.

11. The method of claim 9, wherein the pulse rate threshold is at least 20 beats per minute greater than the user's baseline pulse rate.

12. The method of claim 9, wherein the acceleration is the change in height or elevation of the user followed by inactivity.
